(19) United States
(12) Patent Application Publication
(10) Pub. No.: US 2017/0206995 A1
(43) Pub. Date: Jul. 20, 2017

(54) APPARATUS INCLUDING A BENT INTERFERENCE GRATING AND METHOD FOR BENDING AN INTERFERENCE GRATING FOR INTERFEROMETRIC X-RAY IMAGING

(71) Applicants: Berthold Baumann, Kastl (DE); Alexander Krämer, Irchenrieth (DE); Thomas Weber, Hausen (DE); Josef Zeidler, Marktredwitz (DE)

(72) Inventors: Berthold Baumann, Kastl (DE); Alexander Krämer, Irchenrieth (DE); Thomas Weber, Hausen (DE); Josef Zeidler, Marktredwitz (DE)

(21) Appl. No.: 15/405,568

(22) Filed: Jan. 13, 2017

(30) Foreign Application Priority Data
Jan. 15, 2016 (DE) 102016200440.9

(51) Int. Cl. G21K 1/06; G01N 23/20

(52) U.S. Cl. CPC G21K 1/067; G01N 23/20075; G21K 2201/064; G21K 2201/067; A61B 6/484

(57) ABSTRACT

An apparatus for interferometric x-ray imaging includes an interference grating and a frame-like holding device. The interference grating is bendable like a leaf spring and is arranged in grooves of opposing bearings of the holding device such that the interference grating has one-dimensional concave curvature or one-dimensional convex curvature.

### CROSS-REFERENCE TO RELATED APPLICATIONS

The present patent document claims the benefit of DE 102016200440.9, filed on Jan. 15, 2016, which is hereby incorporated by reference in its entirety.

### TECHNICAL FIELD

The present embodiments relate to interferometric x-ray imaging.

### BACKGROUND

X-ray phase-contrast imaging is an x-ray imaging method that uses the absorption of x-ray radiation through an object as source of information. X-ray phase-contrast imaging combines the absorption of x-ray radiation with a shift in the phase of the x-ray radiation when passing through the object. The information content is great because the absorption of x-ray radiation supplies accurate images of strongly absorbing bones and the phase-contrast supplies sharp images of the structures in the soft tissue. X-ray phase-contrast imaging provides the option of being able to identify pathological changes, such as the creation of tumors, vascular constrictions or pathological changes in cartilage at an earlier stage.

The passage of x-ray radiation through matter is described by a complex refractive index. The imaginary part of the refractive index specifies the strength of the absorption. The real part of the refractive index specifies the phase shift of the x-ray wave passing through a material. In phase-contrast imaging, the phase information about the local phase, or the local gradient of the phase, of the wavefront passing through an object is determined. In a manner analogous to x-ray tomography, tomographic representations of the phase shift may be reconstructed based on a multiplicity of images.

There are a number of options for implementing x-ray phase-contrast imaging. In the known solutions, the focus is placed on making the phase shift of the x-ray radiation visible as an intensity variation as a result of specific arrangements and methods when passing through an object. A very promising method is grating phase-contrast imaging (e.g., Talbot-Lau interferometry) described in the literature (e.g., EP 1 879 020 A1). The main components of the Talbot-Lau interferometer are three x-ray gratings arranged between an x-ray emitter and an x-ray detector.

In addition to the conventional absorption image, such interferometers are able to depict two additional measurement variables in the form of further images: the phase-contrast image; and the dark-field image. The phase of the x-ray wave is determined by interference with a reference wave by using the interferometric grating arrangement.

For example, EP 1 879 020 A1 discloses an arrangement including an x-ray emitter and a pixelated x-ray detector, with an object to be irradiated being arranged between the emitter and the detector. A source grating (e.g., a coherence grating) is arranged between the focal spot of the x-ray tube and the object. The source grating serves to simulate a plurality of line sources with a partial spatial coherence of the x-ray radiation, which is a precondition for interferometric imaging.

A diffraction grating (e.g., a phase grating or Talbot grating) is arranged between the object and the x-ray detector. The diffraction grating impresses a phase shift onto the phase of the wavefront (e.g., typically by pi).

An absorption grating between the diffraction grating and the x-ray detector serves to measure the phase shift generated by the object. The wavefront upstream of the object is “bent” by the object. The three gratings have to be arranged parallel to one another and at exact distances from one another.

The x-ray detector serves for the spatially dependent detection of x-ray quanta. Because the pixelation of the x-ray detector generally does not suffice to resolve the interference strips of the Talbot pattern, the intensity pattern is scanned by shifting one of the gratings (e.g., “phase stepping”). Scanning is carried out act-by-act, or continuously perpendicular to the direction of the x-ray beam and perpendicular to the slit direction of the absorption grating. Three different types of x-ray images may be recorded and reconstructed: the absorption image; the phase-contrast image; and the dark-field image.

The source grating is placed into the x-ray beam when conventional x-ray emitters are used to achieve sufficient transversal coherence for the imaging. On account of the spherical divergence caused by the cone-beam geometry, there is shadowing of the radiation, already at small divergence angles, in the case of plane gratings with a high aspect ratio. A majority of the intensity is absorbed directly behind the source by the source grating. One option for avoiding shadowing by the source grating is use of bent gratings.

Producing bent gratings by virtue of clamping the grating between two bent frame halves, with the curvature at the pressing point of the frame halves generating the required grating curvature, is known from practice. However, no homogeneous curvature may be generated thereby because the inherent stiffness of the gratings leads to the grating springing back. The desired radius of curvature is missed by a large margin, especially at the center of the grating.

Another method for bending is described in DE 10 2006 037 256 A1, with an interference grating bent with the aid of bearing points that are arranged offset from one another.

For the purposes of x-ray imaging, the interference gratings are to each be provided with a predeterminable uniform curvature according to the distance from the focal spot (e.g., focus) of an x-ray emitter to provide a homogeneous image illumination.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an apparatus including a bent interference grating, a phase-contrast imaging device including a bent interference grating, and a method for bending an interference grating with a uniform curvature of the grating for phase-contrast imaging are provided.

According to an embodiment, the apparatus includes a leaf-spring-like interference grating arranged in a frame (e.g., a holding device) such that the interference grating curves in one dimension. By assembling the grating as a “leaf spring” in an integral frame, a homogeneous grating curvature is achieved over the whole length of the grating. The grating curvature over the width of the grating is more homogeneous than in an embodiment with a pressing frame. From a manufacturing point of view, the integral frame is easier to produce, as no complicated clamping surface is required. If the two clamping bearings of the “leaf spring” are configured in a displaceable manner, adjusting the grating curvature is possible (e.g., during assembly). If the displacement of the bearings is embodied in a motor driven manner, a dynamic adjustment of the curvature is possible (e.g., the adjustment following a variable distance from the focal spot).

An apparatus for interferometric x-ray imaging includes a quadrilateral interference grating and a frame-like, quadrilateral holding device. The interference grating has an embodiment that is bendable like a leaf spring and is arranged in opposing bearings of the holding device such that the interference grating has one-dimensional concave curvature or one-dimensional convex curvature. The bearings have grooves in which two opposite side edges of the interference grating are clamped. The bearings are situated in two opposite sides of the holding device.

The embodiments may provide the advantage of the curvature of the interference grating being very homogeneous and the holding device having a planar and simple embodiment.

In a further embodiment, the bearings may be arranged in a displaceable manner such that the curvature of the interference grating is modifiable. As a result, the curvature may easily be adjusted during the adjustment process.

In a further embodiment, a carrier material of the interference grating may be formed from silicon or a ceramic material. As a result, the carrier material is bendable in a very flexible and reversible manner. The active grating structure may be metal or a metal alloy. The carrier material made of silicon or ceramic may be completely removed in a final process act.

In an embodiment, the interference grating may have a thickness of less than 0.5 mm. In other embodiments, the interference grating may be thicker and thinner, for example, if the interference grating is complemented by capping layers (e.g., for protection from ambient influences) that are inactive from a mechanical and x-ray radiation engineering point of view. Thicknesses in the millimeter range may also be provided.

An x-ray phase-contrast imaging device including an x-ray emitter, an x-ray detector, and at least one apparatus according to the present embodiments arranged between the x-ray emitter and the x-ray detector is provided.

In a further embodiment, the device may include an adjustor that has a functional connection with at least one bearing such that the bearing is displaceable via the adjustor.

The adjustor may include an electric motor. As a result of the adjustor, a dynamic adaptation of the curvature of the interference grating may be provided.

A method for bending an interference grating for interferometric x-ray imaging is provided using an apparatus according to one or more of the present embodiments. For example, the bearings are moved toward one another, resulting in the curvature of the interference grating changing.

### BRIEF DESCRIPTION OF THE DRAWINGS

FIG. 1 shows a sectional view of a curved interference grating according to an embodiment.

FIG. 2 shows a plan view of a bent interference grating according to an embodiment.

FIG. 3 shows a spatial view of a bent interference grating according to an embodiment.

FIG. 4 shows an x-ray phase-contrast imaging device according to an embodiment.

### DETAILED DESCRIPTION

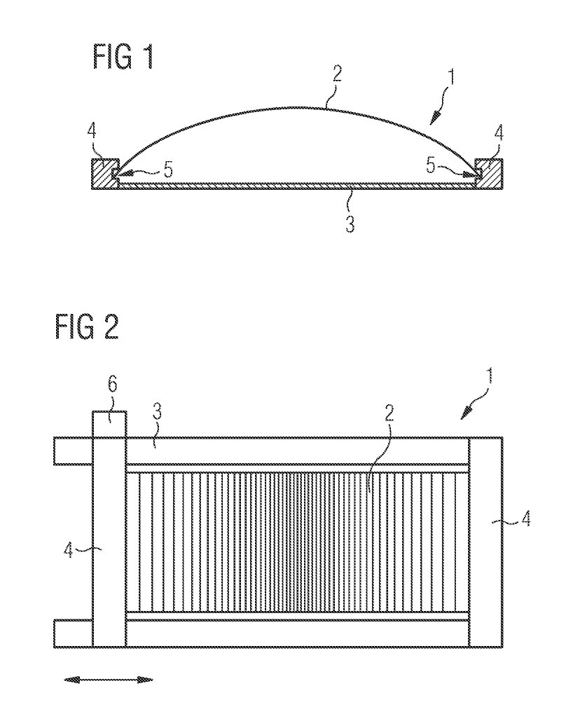

FIG. 1 shows a cross section through an apparatus 1 including a rectangular interference grating 2. The interference grating 2 has a leaf-spring-like embodiment and is clamped in a rectangular, frame-like holding device 3 of the apparatus 1 such that the interference grating 2 has a one-dimensional concave or convex curvature. The two opposite side edges of the interference grating 2 lie in longitudinally arranged grooves 5 of bearings 4 of the holding device 3, and are thus mounted without tension.

The holding device 3 forms a frame with two opposite frame sides each having an interior groove 6, in which the opposite side edges of the interference grating 2 are mounted in a clamped manner. Because the frame is smaller than the interference grating 2, the interference grating 2 arches out of the plane of the frame.

If the length of the interference grating 2 is longer than the distance the bearings 4 are spaced apart from one another, the interference grating 2 arches upward (e.g., if the interference grating 2 has a reversibly bendable material structure). In an embodiment, the interference grating 2 has a thickness between 0.1 and 0 5 mm and the carrier material is formed from a silicon or a ceramic material. The apparatus 1 may have a rectangular embodiment.

FIG. 2 shows a plan view of an apparatus 1 including a bent interference grating 2. The interference grating 2 has a one-dimensional planar upward curvature because the interference grating 2 is loosely clamped in grooves (not visible here) of the bearings 4 of the holding device 3. The left-hand bearing 4 may be displaced in the frame-like holding device 3 in the direction of the arrow, resulting in that the curvature of the interference grating 2 may be modified. The left-hand bearing 4 may be displaced along the side parts of the frame-like holding device 3 with the aid of an electric motor 6 as an adjustor. As a result, the curvature of the interference grating 2 may be configured dynamically.

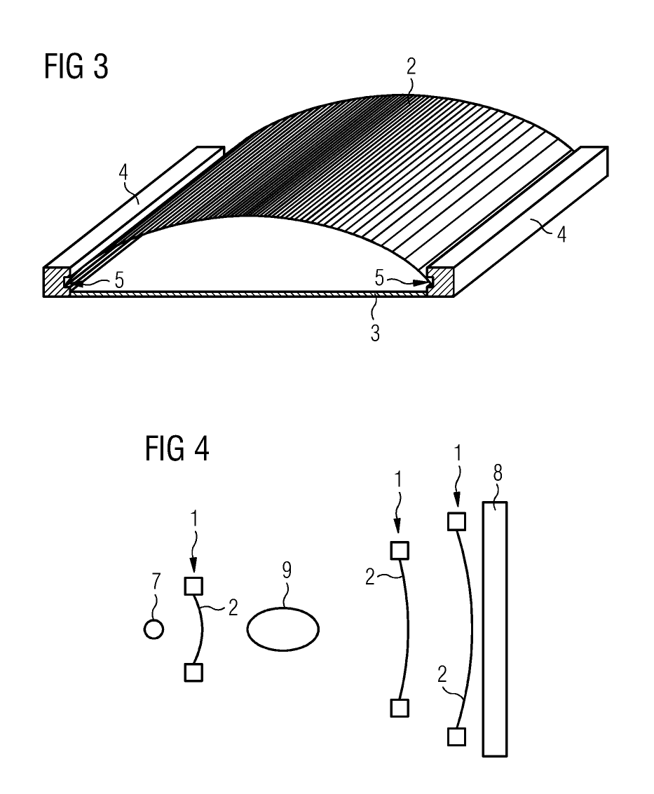

FIG. 3 shows a spatial view of an apparatus 1 including a bent interference grating 2. The interference grating 2 is clamped in grooves 5 of a frame-like holding device 3 and easily bendable on account of the leaf-spring-like properties thereof. The grooves 5 extend in mutually opposite bearings 4 of the holding device 3.

FIG. 4 shows one embodiment of an x-ray phase-contrast imaging device. Situated between an x-ray emitter 7 and an x-ray detector 8 is an object 9 to be irradiated. An interference grating 2 is provided as a source grating upstream of the object 9, and two interference gratings 2 are disposed downstream of the object 9 as phase grating and absorption grating, respectively. The interference gratings 2 are clamped in an apparatus 1 such that the interference gratings 2 have one-dimensional curvature.

Even though the invention was illustrated more closely and described in detail by the exemplary embodiments, the invention is not restricted by the disclosed examples, and other variations may be derived therefrom by a person skilled in the art without departing from the scope of protection of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An apparatus for interferometric x-ray imaging, the apparatus comprising:
an interference grating that is quadrilateral, the interference grating being bendable like a leaf spring; and
a holding device that is frame-like and quadrilateral,
wherein two opposite side edges of the interference grating are clamped in grooves of two opposing bearings of the holding device, such that the interference grating has one-dimensional concave curvature or one-dimensional convex curvature.

2. The apparatus of claim 1, wherein the two opposing bearings are arranged in a displaceable manner such that the curvature of the interference grating is modifiable.

3. The apparatus of claim 1, wherein a carrier material of the interference grating comprises silicon or a ceramic material.

4. The apparatus of claim 1, wherein the interference grating has a thickness of less than 0.5 mm.

5. The apparatus of claim 2, wherein a carrier material of the interference grating comprises silicon or a ceramic material.

6. The apparatus of claim 2, wherein the interference grating has a thickness of less than 0.5 mm.

7. The apparatus of claim 3, wherein the interference grating has a thickness of less than 0.5 mm.

8. An x-ray phase-contrast imaging device comprising:
an x-ray emitter;
an x-ray detector; and
an apparatus for interferometric x-ray imaging arranged between the x-ray emitter and the x-ray detector, the apparatus comprising:
an interference grating that is quadrilateral, the interference grating being bendable like a leaf spring; and
a holding device that is frame-like and quadrilateral,
wherein two opposite side edges of the interference grating are clamped in grooves of two opposing bearings of the holding device such that the interference grating has one-dimensional concave curvature or one-dimensional convex curvature.

9. The x-ray phase-contrast imaging device of claim 8, further comprising an adjustor having a functional connection with at least one bearing of the two opposing bearings such that the at least one bearing is displaceable by the adjustor.

10. The x-ray phase-contrast imaging device of claim 9, wherein the adjustor comprises an electric motor.

11. The x-ray phase-contrast imaging device of claim 8, wherein the two opposing bearings are arranged in a displaceable manner such that the curvature of the interference grating is modifiable.

12. The x-ray phase-contrast imaging device of claim 8, wherein a carrier material of the interference grating comprises silicon or a ceramic material.

13. The x-ray phase-contrast imaging device of claim 8, wherein the interference grating has a thickness of less than 0.5 mm.

14. A method for bending an interference grating for interferometric x-ray imaging using an apparatus comprising a interference grating that is quadrilateral, the interference grating being bendable like a leaf spring, the apparatus further comprising a holding device that is frame-like and quadrilateral, wherein two opposite side edges of the interference grating are clamped in grooves of two opposing bearings of the holding device such that the interference grating has one-dimensional concave curvature or one-dimensional convex curvature, the method comprising:
moving the two opposing bearings of the holding device toward one another, the moving of the two opposing bearings changing the curvature of the interference grating.
